(19) United States
(12) Patent Application Publication
(10) Pub. No.: US 2011/0136195 A1
(43) Pub. Date: Jun. 9, 2011

(54) GENETICALLY-ENGINEERED YEAST AND METHODS OF MAKING AND USING

(75) Inventors: Glenn Richards, Visalia, CA (US); Adriano Galvez, III, Visalia, CA (US)

(73) Assignee: EdeniQ, Inc., Visalia, CA (US)

(21) Appl. No.: 13/059,054

(22) PCT Filed: Aug. 14, 2009
(86) PCT No.: PCT/US09/53886

§ 371 Date: Feb. 14, 2011

Related U.S. Application Data
(60) Provisional application No. 61/089,225, filed on Aug. 15, 2008.

(51) Int. Cl. C12P 7/06; C12N 1/19; C12N 15/74

(52) U.S. Cl. 435/161; 435/254.21; 435/471

(57) ABSTRACT

This disclosure describes genetically-engineered yeast that are able to uptake glycerol and convert the glycerol into ethanol.

### CROSS-REFERENCE

The present patent application claims benefit of priority to U.S. Provisional Patent Application No. 61/089,225, filed Aug. 15, 2008, which is incorporated by reference for all purposes.

### TECHNICAL FIELD

This disclosure relates to genetically-engineered yeast.

### BACKGROUND

Yeast were originally developed for making wines and beers by selection of naturally occurring variants that produce other products of value including glycerol (also known as glycerin), succinic acid and additional metabolic organic compounds. Glycerol, in some alcoholic beverages, is a requirement to get a particular taste and feel. In fact, substantial efforts were made to find additives to fermentation media that would drive the level of glycerol higher.

With the advent of ethanol fuel, natural selection of yeast strains, improved yeast nutrition, and the production of high starch corn hybrids has led to higher sugar utilization, which results in improved yields. For example, ethanol concentrations of 12-14 wt % can be obtained at commercial production facilities. Although production levels of ethanol from corn continued to grow substantially, there is little difference in the various yeasts available and used in commercial fermentations.

With the production of corn ethanol and other biomass to biofuels applications came an enormous spike in the production of glycerol. The high cost of energy and the desire to maximize plant capacity has led to an increase in the weight percent of solids from levels of 26% to over 34% in fermentation. As the amount of solids increases, the production of glycerol continues to grow ultimately leading to an increase of this by-product in the Distillers Dried Grains w/ Solubles (DDGS). Additionally, glycerol is a by-product of biodiesel production that can reach up to 10% by weight. All of these factors contribute to the need for genetically-engineered yeast that are able to convert glycerol to ethanol.

### SUMMARY

This disclosure describes genetically-engineered yeast that are able to uptake glycerol and convert the glycerol into ethanol.

In one aspect, a yeast comprising a first heterologous nucleic acid sequence, a second heterologous nucleic acid sequence, a third heterologous nucleic acid sequence, and a fourth heterologous nucleic acid sequence is provided. As described herein, the first nucleic acid sequence results in the expression of a protein that increases the amount or activity of a polypeptide that is able to transport glycerol into the yeast cell. Expression of the second nucleic acid sequence results in an increase in the amount or activity of a second polypeptide that is able to convert glycerol into dihydroxyacetone (DHA). The third nucleic acid sequence results in the increased expression or activity of a polypeptide that is able to phosphorylate DHA to produce dihydroxyacetone phosphate (DHAP) and the fourth nucleic acid sequence results in a the expression of a protein that increases the amount or activity of a fourth polypeptide that is able to convert DHAP into glycereroldehyde-3-phosphase (GAP).

Representative polypeptides from the first group of nucleic acid sequences include a polyol transporter, a sugar transporter or a glycerol transporter. In certain instances, the second polypeptide has glycerol dehydrogenase activity; the third polypeptide has dihydroxyacetone kinase activity; and the fourth polypeptide has triose-phosphate isomerase activity.

In some embodiments, the first polypeptide can be encoded by a polynucleotide substantially identical to a polypeptide encoded by a polynucleotide selected from the group consisting of SEQ ID NO: 4, 5, and 6. In certain instances, the polypeptide having glycerol dehydrogenase activity is substantially identical to SEQ ID NO:13 or the polypeptide that is encoded by a S. cerevisiae nucleic acid designated GCY1 (GenBank Accession Numbers CAA99318 and CAA65512). In some embodiments, the polypeptide having dihydroxyacetone kinase activity is substantially identical to SEQ ID NO:15, 16, or 17 or to the polypeptide that is encoded by S. cerevisiae nucleic acids designated DAK1 and/or DAK2 (GenBank Accession Numbers NP_{—}013641 and NP_{—}116602). In some embodiments, the polypeptide having triosephosphate isomerase activity is substantially identical to SEQ ID NO:14 or to the polypeptide that is encoded by a S. cerevisiae nucleic acid designated TPI (GenBank Accession Number AAA88757.1). Typically, the yeast described herein, under fermentation conditions, will produce reduced (e.g., reduced by at least 5%, 10%, 20% or more) amounts of glycerol and/or increased (e.g., increased by at least 5%, 10%, or more) amounts of ethanol compared to, for example, yeast lacking a corresponding first, second, third, and/or fourth nucleic acid sequence. In still another aspect, methods of producing ethanol are provided. Such methods typically include contacting any of the yeasts described herein with biomass or with glycerol.

Also provided in this disclosure are methods of making a genetically-engineered yeast of the invention. Such methods typically include introducing a first nucleic acid sequence, a second nucleic acid sequence, a third nucleic acid sequence, and a fourth nucleic acid sequence into a yeast. In certain instances, the first nucleic acid encodes a first polypeptide that is able to transport glycerol into the yeast cell; the second nucleic acid sequence encodes a second polypeptide that is able to convert glycerol into dihydroxyacetone (DHA); the third nucleic acid sequence encodes a third polypeptide that is able to phosphorylate DHA to produce dihydroxyacetone phosphate (DHAP); and the fourth nucleic acid sequence encodes a fourth polypeptide that is able to convert DHAP into glycereroldehyde-3-phosphase (GAP). In certain instances, the first nucleic acid sequence encodes a polyol transporter, a sugar transporter or a glycerol transporter; the second nucleic acid sequence encodes a polypeptide having glycerol dehydrogenase activity; the third nucleic acid sequence encodes a polypeptide having dihydroxyacetone kinase activity; and the fourth nucleic acid sequence encodes a polypeptide having triose-phosphate isomerase activity.

Yeast produced by such methods can produce less glycerol while making more ethanol than corresponding yeast lacking the first, second, third and fourth nucleic acid sequences. In certain instances, the first, second, third, and/or fourth nucleic acid sequences can be integrated into the yeast chromosome. The yeast described herein can be S. cerevisiae or any other species of yeast.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a schematic showing a number of enzymatic pathways in yeast.

FIG. 2 shows the nucleic acid sequences used in the constructs described herein.

FIG. 3 shows the amino acid sequences of the polypeptides that will be expressed in the genetically-engineered yeast described herein.

### DETAILED DESCRIPTION

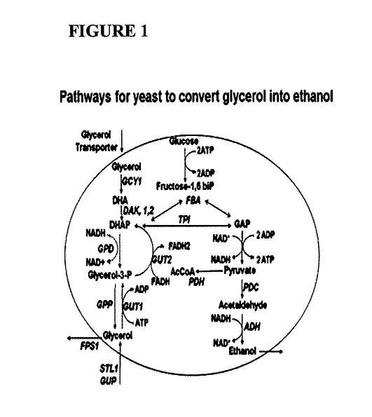

Yeast are described herein that will be able to convert glycerol into ethanol. The yeast described herein will be genetically-engineered to transport glycerol into the cell and efficiently convert the glycerol to glyceraldehyde-3-phosphate (GAP). GAP then can be used by the yeast in existing pathways to produce ethanol. See, for example, FIG. 1. Any type of yeast can be genetically-engineered as described herein, including but not limited to, Saccharomyces cerevisiae, S. bayanus, S. pastorianus, and Pichia stipidis.

The genetically-engineered yeast described herein will be used to convert glycerol to ethanol during the fermentation of any type of biomass. As used herein, biomass includes, but is not limited to, crops such as starch crops (e.g., corn, wheat, rice or barley), sugar crops (e.g., sugarcane, energy cane or sugarbeet), forage crops (e.g., grasses, alfalfa, or clover), and oilseed crops (e.g., soybean, sunflower, or safflower); wood products such as trees, shrubs, and wood residues (e.g., sawdust, bark or the like from forest clearings and mills); waste products such as municipal solid waste (MSW; e.g., paper, food and yard wastes or wood), and process waste; and aquatic plants such as algae, water weed, water hyacinth, or reed and rushes.

Polypeptides that Transport Glycerol

There are a number of polypeptides that are able to transport glycerol into a yeast cell. For example, polyol transporters (e.g., polyol transporter (PLT) 1 and 2 from Plantago major (PmPLT1 and PmPLT2)), sugar transporters (e.g., MfsX from Erwinia chrysanthemi, and STL1 encoding Stl1p from S. cerevisiae), or glycerol transporters (e.g., glycerol uptake 1 & 2 [GUP1 (SEQ ID NO: 18, or as encoded by SEQ ID NO:8), GUP2 (Seq ID NO:19, or as encoded by SEQ ID NO:9)], and CrMIP1 from Chlamydomonas reinhardtii) have been identified.

Representative nucleic acid sequences encoding polypeptides that are able to transport glycerol into a cell include, for example, a polypeptide substantially identical to the proteins encoded by, e.g., GenBank Accession No XM_{—}571264. Representative amino acid sequences of polypeptides that are able to transport glycerol into a cell include, without limitation, polypeptides substantially identical to NP_{—}188513; CAJ29291; AAB68029; ZP_{—}01877964; EDM33348; ABA78818; EDN62033; and EDN60957.

### Conversion of Glycerol to Glyceraldehyde-3-Phosphate

Once glycerol is taken up into the cell, it can be converted into dihydroxyacetone (DHA) using an aldo/keto reductase such as glycerol dehydrogenase (e.g., designated GCY1 (SEQ ID NO:13) in S. cerevisiae). Polypeptides having glycerol dehydrogenase activity are assigned to Enzyme Classification (EC) 1.1.99.22 under the IUBMB Enzyme Nomenclature system. Representative nucleic acid sequences encoding polypeptides having glycerol dehydrogenase activity include, for example, those sequences shown in GenBank Accession Nos. EU679508; NM_{—}001019083; XM_{—}743542; XM_{—}001275320; DQ985161; DQ641687; and AB185335. Representative amino acid sequences of polypeptides having glycerol dehydrogenase activity (e.g., that are able to convert glycerol to DHA) include, without limitation, those sequences shown in GenBank Accession Nos. EDP56229; EDP53335; XP_{—}752360; XP_{—}748635; EAL90322; EAL86597; XP_{—}001269267; EAW22877; and EAW07841. Nucleic acids encoding polypeptides having glycerol dehydrogenase activity have been identified from a number of genera and species including, but not limited to, Aspergillus fumigates, Aspergillus clavatus, Aspergillus niger, Neosartorya fisheri and Pichia stipidis. Glycerol dehydrogenase sequences within a genera exhibit very high sequence identity (e.g., >99% in Aspergillus). Thus, exemplary polypeptides of that converts glycerol to DHA include those identical or substantially identical to a polypeptide encoded by any of the sequences above.

The resulting DHA then can be phosphorylated with a kinase to produce dihydroxyacetone phosphate (DHAP). Any kinase capable of phosphorylating DHA to DHAP can be used according to the invention. Suitable kinases include dihydroxyacetone kinase (DAK; also known as glycerone kinase (SEQ ID NOS: 15, 16, or 17)). Polypeptides having dihydroxyacetone kinase activity are assigned to Enzyme Classification (EC) 2.7.1.29 under the IUBMB Enzyme Nomenclature system. Representative nucleic acid sequences encoding dihydroxyacetone kinase sequences include, without limitation, those sequences shown in GenBank Accession Nos. BC081941; XM_{—}718181; NM_{—}112658; NM_{—}001018638; NM_{—}001018187; and NM_{—}015533. Representative amino acid sequences of dihydroxyacetone kinase polypeptides include, for example, those sequences shown in GenBank Accession Nos. NP_{—}056348; YP_{—}001070872; and ZP_{—}02837587. Thus, exemplary polypeptides of that converts DHA to DHAP include those identical or substantially identical to a polypeptide encoded by any of the sequences above.

Nucleic acid sequences encoding DAK polypeptides have been identified in a number of genera and species including Mycobacterium, Rhizobium and Arthrobacter. In S. cerevisiae, the nucleic acid sequences encoding DAK are segmented into two functional groups; DAK1, an isoform involved in carbohydrate transport and metabolism, and DAK2, an isoform the kinase domain. DAKs exhibit significantly high sequence identity (>99%), even across genera and species. Either DAK1 or DAK2 isoform is believed to be useful in the present invention.

DHAP then can be converted to glyceraldehydes-3-phosphate (GAP) using an isomerase such as triose-phosphate isomerase (TPI; also known as TIM or TPIM). Polypeptides having TPI activity are assigned to Enzyme Classification (EC) 5.3.1.1 under the IUBMB Enzyme Nomenclature system. Representative nucleic acid sequences encoding TPI polypeptides can be found, without limitation, in the sequences shown in GenBank Accession Nos. NM_{—}001097154; NM_{—}000365; BC102903; NM_{—}127687; and NM_{—}001126258. Representative amino acid sequences of TPI polypeptides can be found in, for example, the sequences shown in GenBank Accession Nos. ABM47443; AAC13160; ABM47435; and CAD99190. Thus, exemplary polypeptides of that converts DHAP to GAP include those identical or substantially identical to a polypeptide encoded by any of the sequences above.

Nucleic acid sequences encoding TPI polypeptides have been identified from a number of genera including Plethodon, Clostridium, Lactobacillus and Aneides. The sequence identity among TPI nucleic acids/polypeptides across genera and species typically is greater than 90%. Nucleic acid sequences encoding the polypeptides described herein are sufficient to bring glycerol into a cell and convert the glycerol to ethanol.

Various proteins involved in glycerol transport and metabolism are described in, for example, Nevoigt and Stahl, FEMS Microbiol. Review 21: 231-241 (1997).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., at least 60% identity, optionally at least 65%, 70%, 75%, 80%, 85%, 90%, 95%, 98%, or 99% identity over a specified region (or the whole reference sequence when not specified)), when compared and aligned for maximum correspondence over a comparison window, or designated region as measured using one of the following sequence comparison algorithms or by manual alignment and visual inspection. Sequences having less than 100% identity but that have at least one of the above-specified percentages are said to be “substantially identical.” Optionally, the identity exists over a region that is at least about 50 nucleotides in length, or more preferably over a region that is 100 to 500 or 1000 or more nucleotides in length.

The term “similarity,” or “percent similarity,” in the context of two or more polypeptide sequences, refer to two or more sequences or subsequences that have a specified percentage of amino acid residues that are either the same or similar as defined in the 8 conservative amino acid substitutions defined above (i.e., 60%, optionally 65%, 70%, 75%, 80%, 85%, 90%, or 95% similar over a specified region), when compared and aligned for maximum correspondence over a comparison window, or designated region as measured using one of the following sequence comparison algorithms or by manual alignment and visual inspection. Sequences having less than 100% similarity but that have at least one of the specified percentages are said to be “substantially similar.” Optionally, this identity exists over a region that is at least about 50 amino acids in length, or more preferably over a region that is at least about 100 to 500 or 1000 or more amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Examples of an algorithm that is suitable for determining percent sequence identity and sequence similarity include the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes or other nucleic acid sequences arranged to make a new functional nucleic acid, e.g., a promoter from one source and a coding region from another source.

An “expression cassette” is a nucleic acid construct, generated recombinantly or synthetically, with a series of specified nucleic acid elements that permit transcription of a particular nucleic acid in a host cell. The expression cassette can optionally be part of a plasmid, virus, or other nucleic acid fragment. Typically, the expression cassette includes a nucleic acid to be transcribed operably linked to a promoter.

### Nucleic Acids and Polypeptides

There are a number of ways in which the amount or activity of a polypeptide can be increased. The amount of a polypeptide can be increased by expressing or over-expressing a nucleic acid sequence encoding the polypeptide. In order to express or over-express a nucleic acid sequence, the number of copies of a nucleic acid sequence can be increased; a nucleic acid sequence can be genetically engineered so as to be expressed under a different or stronger promoter and/or enhancer; the promoter and/or other regulatory elements of a nucleic acid sequence can be altered so as to direct high levels of expression (e.g., the binding strength of a promoter region for its transcriptional activators can be increased); the half-life of the transcribed mRNA can be increased; or the degradation of the mRNA and/or polypeptide can be inhibited. The activity of a polypeptide can be increased, for example, by genetically modifying a nucleic acid sequence such that one or more activities of the encoded polypeptide (e.g., rate of conversion, affinity for substrate) is increased. An amount of a polypeptide is considered to be increased when the polypeptide is present at levels that are at least 20% higher than the levels of polypeptide present in wild type yeast. The activity of a polypeptide is considered to be increased when the polypeptide exhibits at least two-fold greater activity than that exhibited by a wild type polypeptide.

One or more copies of a nucleic acid sequence to be expressed or over-expressed can be expressed from an extrachromosomal vector (also referred to as a construct or plasmid), or one or more copies of a nucleic acid sequence to be expressed or over-expressed can be integrated into the yeast chromosome. Constructs suitable for expressing or over-expressing a nucleic acid are commercially available (e.g., expression vectors) or can be produced by recombinant DNA technology methods routine in the art. See, for example, Akada et al. (2002, Yeast, 19:17-28; and Mitchell et al. (1993, Yeast, 9:715-22). In addition, methods for stably integrating nucleic acid into the yeast genome are known and routine in the art. See, for example, Methods in Enzymology: Guide to Yeast Genetics and Molecular Biology, Vol. 194, 2004, Abelson et al., eds., Academic Press.

A construct containing a nucleic acid sequence can have elements necessary for expression linked to a nucleic acid sequence that encodes a polypeptide, and further can include sequences such as those encoding a selectable marker (e.g., an antibiotic resistance gene, auxotrophic marker), and/or those that can be used in purification of the polypeptide (e.g., 6×His tag). A construct can contain a nucleic acid sequence encoding a polypeptide, or a construct can contain more than one nucleic acid sequence encoding more than one polypeptide. A construct also can include one or more origins of replication.

Elements necessary for expression include nucleic acid sequences that direct and regulate expression of nucleic acid coding sequences. One example of an element necessary for expression is a promoter sequence. Representative promoters include, without limitation, a promoter from a gene encoding a phosphoglycerate kinase, a promoter from a gene encoding a histone acetyltransferase (HAT), and a promoter from a gene encoding a cytochrome C. Elements necessary for expression also can include intronic sequences, enhancer sequences, response elements, or inducible elements that modulate expression of a nucleic acid coding sequence.

Elements necessary for expression can be of bacterial, yeast, insect, plant, mammalian, fungal, or viral origin, and vectors or constructs can contain a combination of elements from different origins. Elements necessary for expression are described, for example, in Goeddel, 1990, Gene Expression Technology: Methods in Enzymology, 185, Academic Press, San Diego, Calif. As used herein, operably linked means that a promoter and/or other regulatory element(s) are positioned in a construct relative to a nucleic acid sequence encoding a polypeptide in such a way as to direct or regulate expression of the nucleic acid sequence. In certain instances, the nucleic acid sequences and/or the elements necessary for expression may be codon optimized to obtain optimal expression in yeast. See, for example, Bennetzen & Hall, 1982, J. Biol. Chem., 257:3026-31.

Nucleic acid sequences (e.g., expression vectors) can be introduced into yeast cells or other host cells using any of a number of different methods. Such methods include, without limitation, electroporation, calcium phosphate precipitation, heat shock, lipofection, microinjection, lithium chloride, lithium acetate, β-mercaptoethanol, and viral-mediated nucleic acid transfer. “Host cells” can include, in addition to yeast cells, cells that can be used in standard molecular biology techniques to manipulate and produce the nucleic acids and polypeptides described herein. “Host cells” include, without limitation, bacterial cells (e.g., E. coli), insect cells, plant cells or mammalian cells (e.g., CHO or COS cells). “Yeast cells,” including the genetically-engineered yeast cells described herein, and other types of “host cells” refers, not only to the particular cell(s) into which a nucleic acid sequence was introduced, but also to the progeny of such cells.

As used herein, an “isolated” nucleic acid molecule (represented by a nucleic acid sequence) is a nucleic acid molecule that is separated from other nucleic acid molecules that are usually associated with the reference nucleic acid molecule in the genome. Thus, an “isolated” nucleic acid molecule includes, without limitation, a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a construct (e.g., a cloning vector, or an expression vector) for convenience of manipulation or to express a fusion polypeptide. In addition, an isolated nucleic acid molecule can include an engineered nucleic acid molecule such as a recombinant or a synthetic nucleic acid molecule.

Nucleic acids can be obtained using techniques routine in the art. For example, isolated nucleic acids can be obtained using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid molecule. Isolated nucleic acids also can be chemically synthesized, either as a single nucleic acid molecule or as a series of oligonucleotides. In addition, isolated nucleic acids also can be obtained by mutagenesis.

Amplification of nucleic acids can be used to produce or detect a nucleic acid. Conditions for amplification of a nucleic acid and detection of an amplification product are known to those of skill in the art (see, e.g., PCR Primer: A Laboratory Manual, 1995, Dieffenbach & Dveksler, Eds., Cold Spring Harbor Laboratory Press, Cold Spring Harbor, N.Y.; and U.S. Pat. Nos. 4,683,195; 4,683,202; 4,800,159; and 4,965,188). Modifications to the original PCR also have been developed. For example, anchor PCR, RACE PCR, or ligation chain reaction (LCR) are additional PCR methods known in the art (see, e.g., Landegran et al., 1988, Science, 241:1077 1080; and Nakazawa et al., 1994, Proc. Natl. Acad. Sci. USA, 91:360 364).

Hybridization of nucleic acids also can be used to obtain or detect a nucleic acid. Hybridization between nucleic acid molecules is discussed in detail in Sambrook et al. (1989, Molecular Cloning: A Laboratory Manual, 2nd Ed., Cold Spring Harbor Laboratory Press, Cold Spring Harbor, N.Y.; Sections 7.37-7.57, 9.47-9.57, 11.7-11.8, and 11.45-11.57). For oligonucleotide probes less than about 100 nucleotides, Sambrook et al. discloses suitable Southern blot conditions in Sections 11.45-11.46. The Tm between a sequence that is less than 100 nucleotides in length and a second sequence can be calculated using the formula provided in Section 11.46. Sambrook et al. additionally discloses prehybridization and hybridization conditions for a Southern blot that uses oligonucleotide probes greater than about 100 nucleotides (see Sections 9.47-9.52). Hybridizations with an oligonucleotide greater than 100 nucleotides generally are performed 15-25° C. below the Tm. The Tm between a sequence greater than 100 nucleotides in length and a second sequence can be calculated using the formula provided in Sections 9.50-9.51 of Sambrook et al. Additionally, Sambrook et al. recommends the conditions indicated in Section 9.54 for washing a Southern blot that has been probed with an oligonucleotide greater than about 100 nucleotides.

The conditions under which membranes containing nucleic acids are prehybridized and hybridized, as well as the conditions under which membranes containing nucleic acids are washed to remove excess and non-specifically bound probe can play a significant role in the stringency of the hybridization. Such hybridizations and washes can be performed, where appropriate, under moderate or high stringency conditions. Such conditions are described, for example, in Sambrook et al. section 11.45-11.46. For example, washing conditions can be made more stringent by decreasing the salt concentration in the wash solutions and/or by increasing the temperature at which the washes are performed. In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium.

It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane. A nucleic acid molecule is deemed to hybridize to a target nucleic acid but not to a non-target nucleic acid if hybridization to a target nucleic acid is at least 5-fold (e.g., at least 6-fold, 7-fold, 8-fold, 9-fold, 10-fold, 20-fold, 50-fold, or 100-fold) greater than hybridization to a non-target nucleic acid. The amount of hybridization can be quantitated directly on a membrane or from an autoradiograph using, for example, a Phosphorlmager or a Densitometer (Molecular Dynamics, Sunnyvale, Calif.).

The term “purified” polypeptide (or protein) as used herein refers to a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated. Since a polypeptide that is chemically synthesized is, by nature, separated from the components that naturally accompany it, a synthetic polypeptide always would be considered “purified.”

Polypeptides can be purified from natural sources (e.g., a biological sample) by known methods such as DEAE ion exchange, gel filtration, and hydroxyapatite chromatography. A purified polypeptide also can be obtained, for example, by expressing a nucleic acid molecule in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis. As described elsewhere in this disclosure, polypeptides can be produced using recombinant expression vectors or constructs.

Antibodies can be used to detect the presence or absence of polypeptides. Techniques for detecting polypeptides using antibodies include enzyme linked immunosorbent assays (ELISAs), Western blots, immunoprecipitations and immunofluorescence. An antibody can be polyclonal or monoclonal, and usually is detectably labeled. An antibody having specific binding affinity for a polypeptide can be generated using methods well known in the art. The antibody can be attached to a solid support such as a microtiter plate using methods known in the art (see, for example, Leahy et al., 1992, BioTechniques, 13:738-743). In the presence of an appropriate polypeptide, an antibody-polypeptide complex is formed.

Detection of an amplification product, a hybridization complex, or a polypeptide-antibody complex usually is accomplished using detectable labels. The term “labeled” with regard to an agent (e.g., an oligonucleotide, a polypeptide, or an antibody) is intended to encompass direct labeling of the agent by coupling (i.e., physically linking) a detectable substance to the agent, as well as indirect labeling of the agent by reactivity with another reagent that is directly labeled with a detectable substance. Detectable substances include various enzymes, prosthetic groups, fluorescent materials, chemoluminescent materials, bioluminescent materials, and radioactive materials.

### Methods of Using Yeast Strains

The genetically-engineered yeast described herein or genetically-engineered yeast made using the methods described herein can be used in fermentation reactions to take up glycerol and convert the glycerol to ethanol. Increasing the ability of yeast to uptake glycerol into the cell and convert that glycerol into ethanol provides means, not only for converting the glycerol by-product produced by yeast during ethanol production into ethanol, but also for converting exogenously added glycerol into ethanol. Therefore, the glycerol that is converted to ethanol by the genetically-engineered yeast described herein can be endogenous (e.g., produced intracellularly by the yeast) or exogenous (e.g., not produced by the yeast; provided extracellularly). For example, the glycerol produced during biodiesel production can be added to a fermentation reaction in the presence of yeast genetically-engineered as described herein.

The genetically-engineered yeast described herein exhibits high ethanol tolerance and can be used in fermenters having a solid content of greater than 34%. In addition, because the genetically-engineered yeast described herein are able to convert glycerol to ethanol, less glycerol is produced in the fermenter and, thus, in the byproduct streams. The reduced levels of glycerol results in reduced energy loads on the DDGS dryers and reduced fire hazards in the shipping of the DDGS. The additional growth conditions (e.g., temperature, pH, agitation, and/or oxygenation) for a genetically-engineered yeast as described herein can be determined using routine experimentation.

The genetically-engineered yeast described herein can be utilized in a variety of feedstocks including corn, corn stover, energy cane and other biomass materials commonly used for the fermentation of starch products into ethanol. This yeast will typically be able to operate in the temperature ranges of 25 C to 37 C. In some embodiments, solids to be fermented can range from as low as 10% to as high as 40% material on a weight basis.

In accordance with the present disclosure, there may be employed conventional molecular biology, microbiology, biochemical, and recombinant DNA techniques within the skill of the art. Such techniques are explained fully in the literature. The methods and compositions of matter are further described in the following examples, which do not limit the scope of the claims.

### EXAMPLES

### Example 1

### Genetically-Engineered Yeast for Uptake and Conversion of Glycerol to Ethanol

Yeast constructs can be generated that contain the nucleic acid sequences necessary to express the various genes encoding proteins that are required for the transport and conversion of glycerol into ethanol.

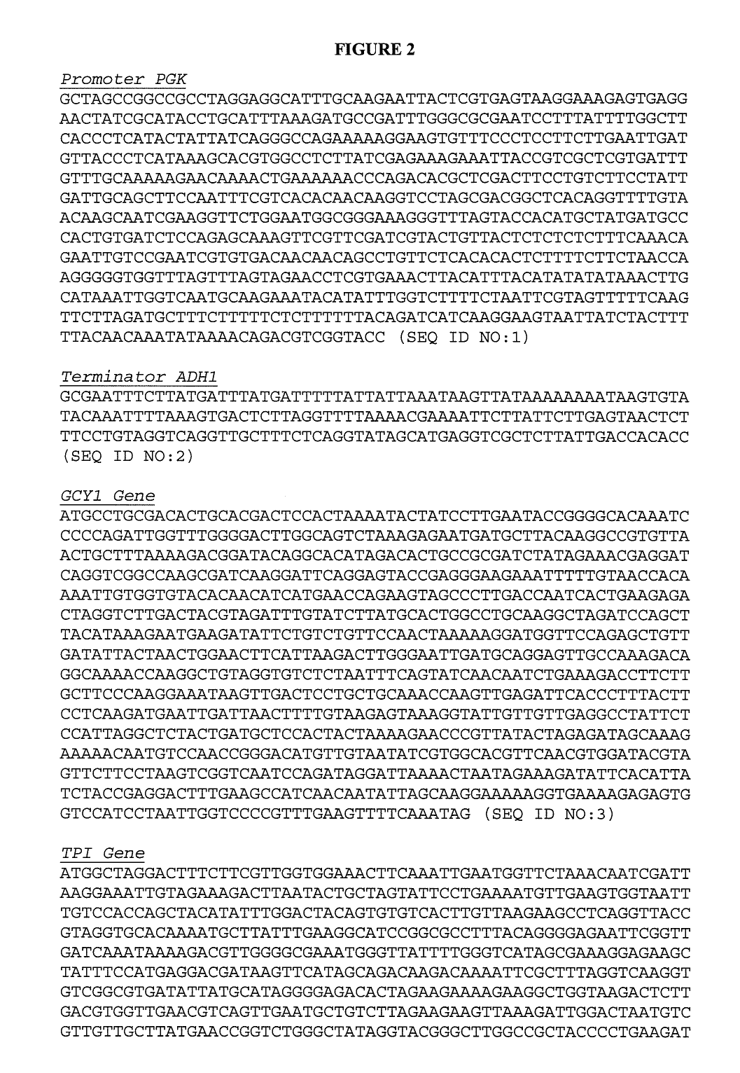

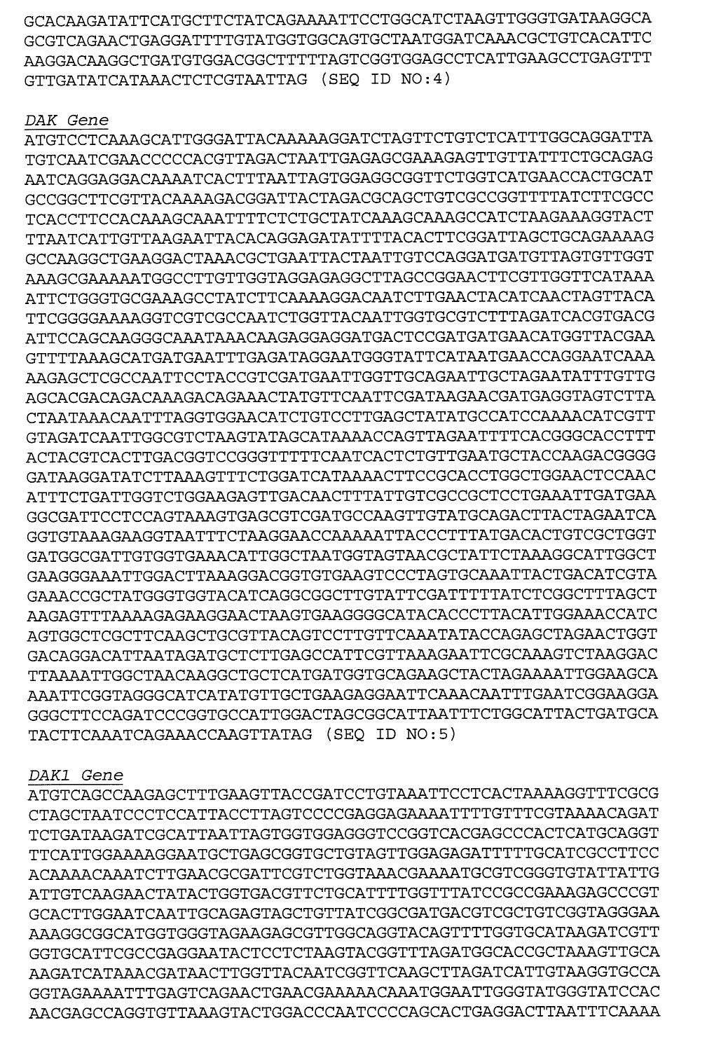

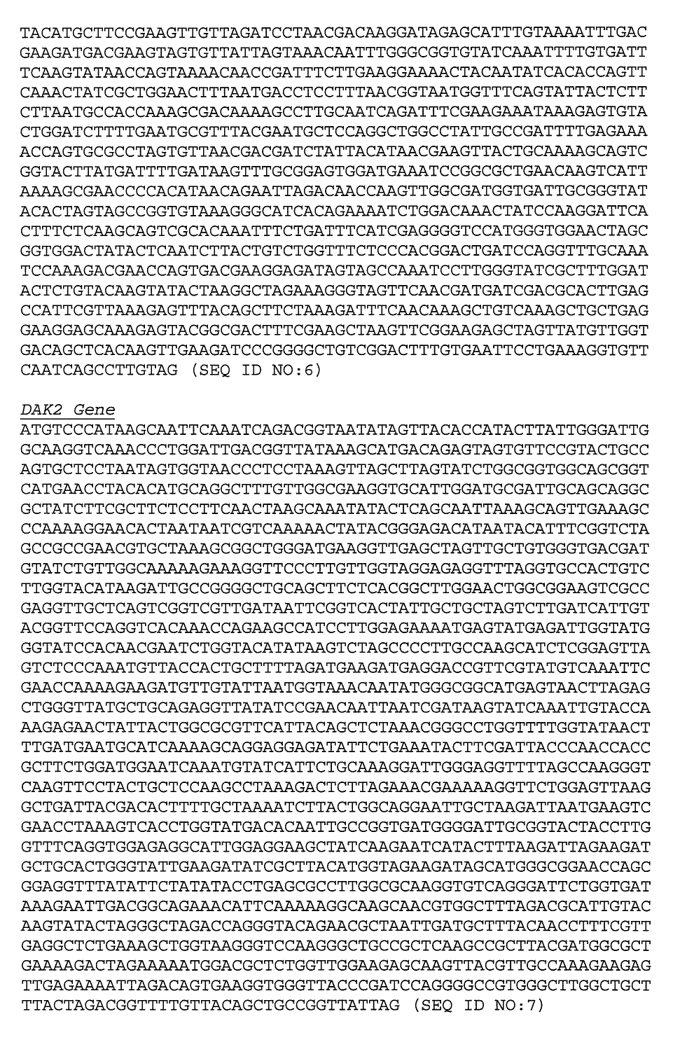

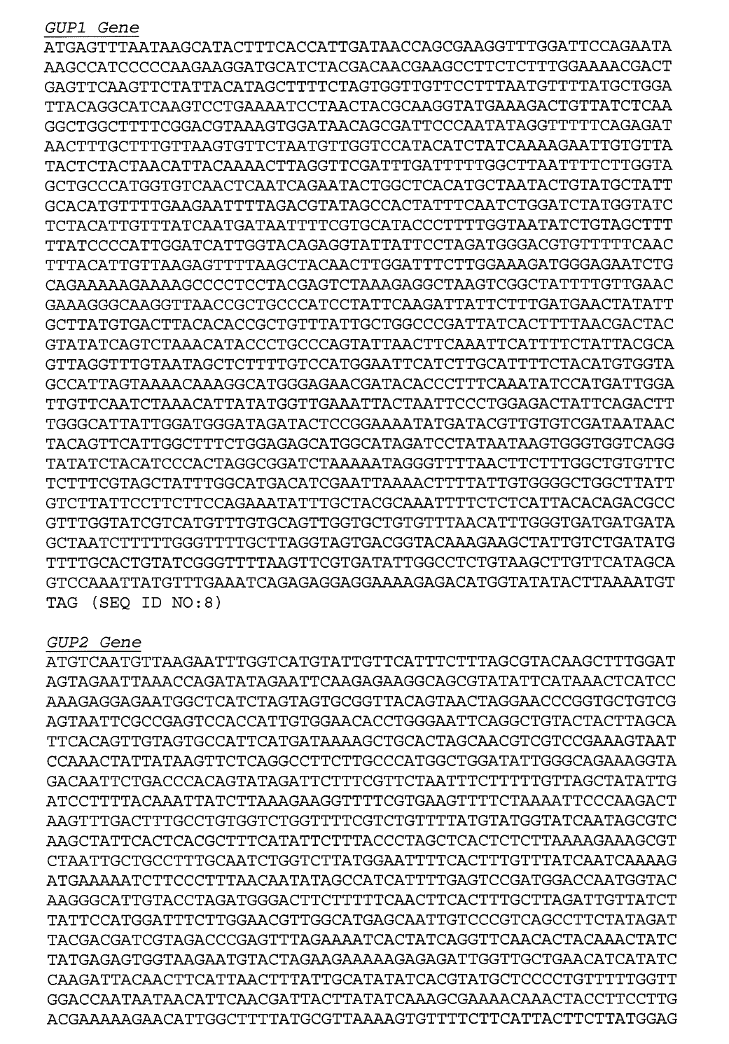

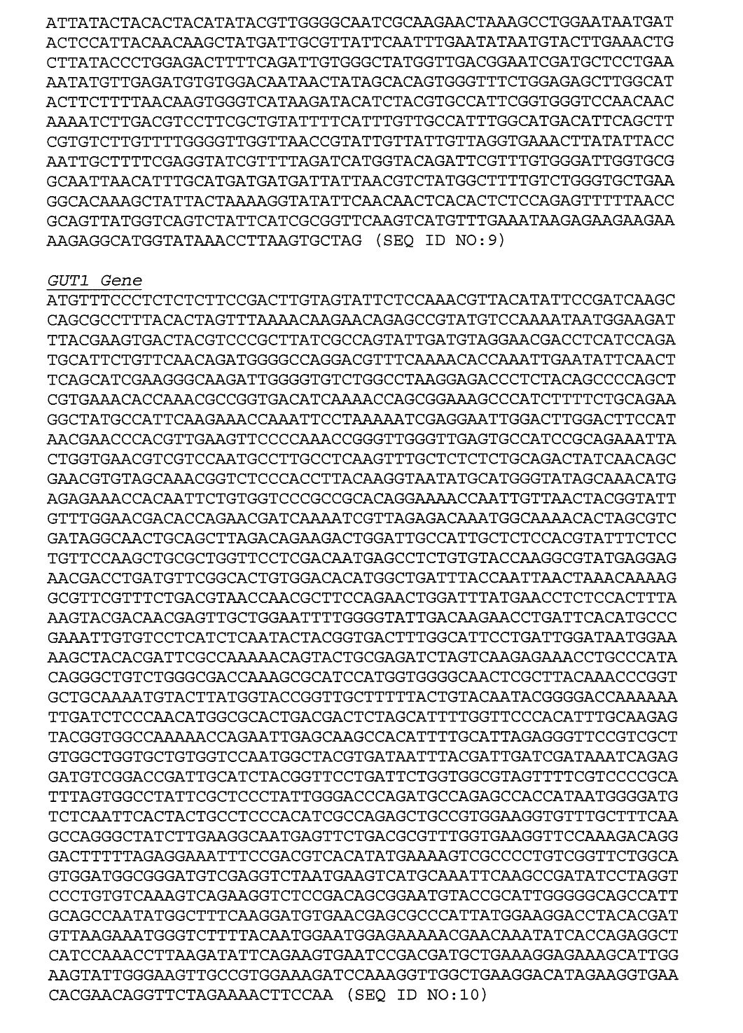

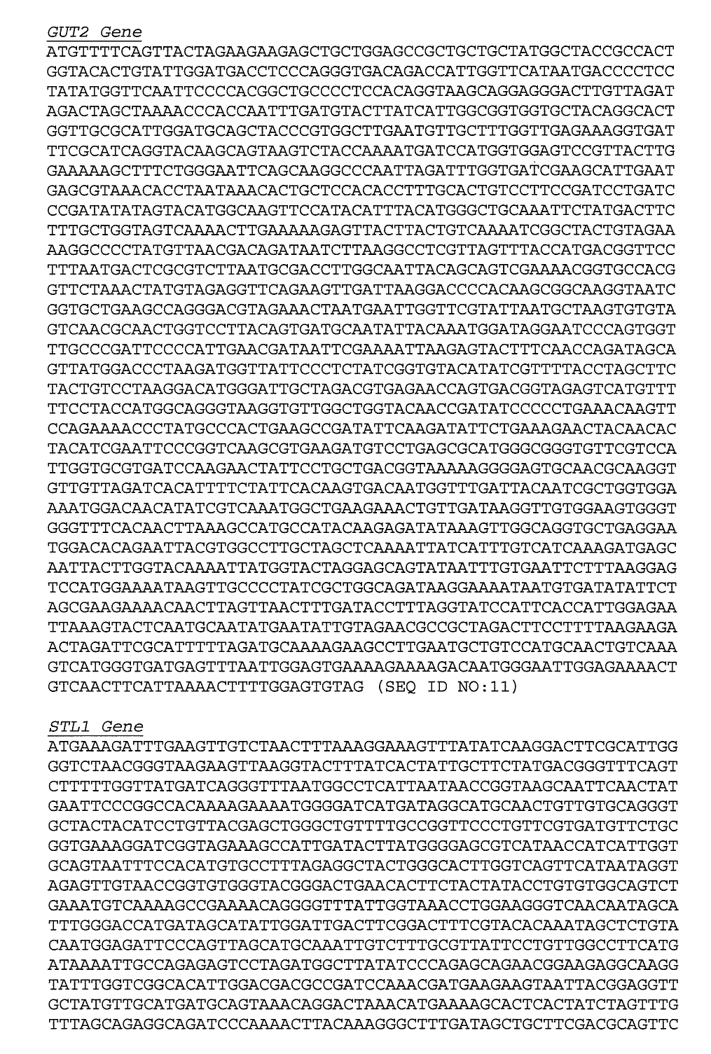

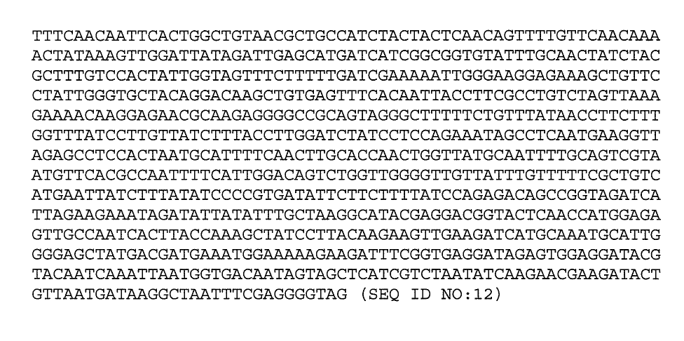

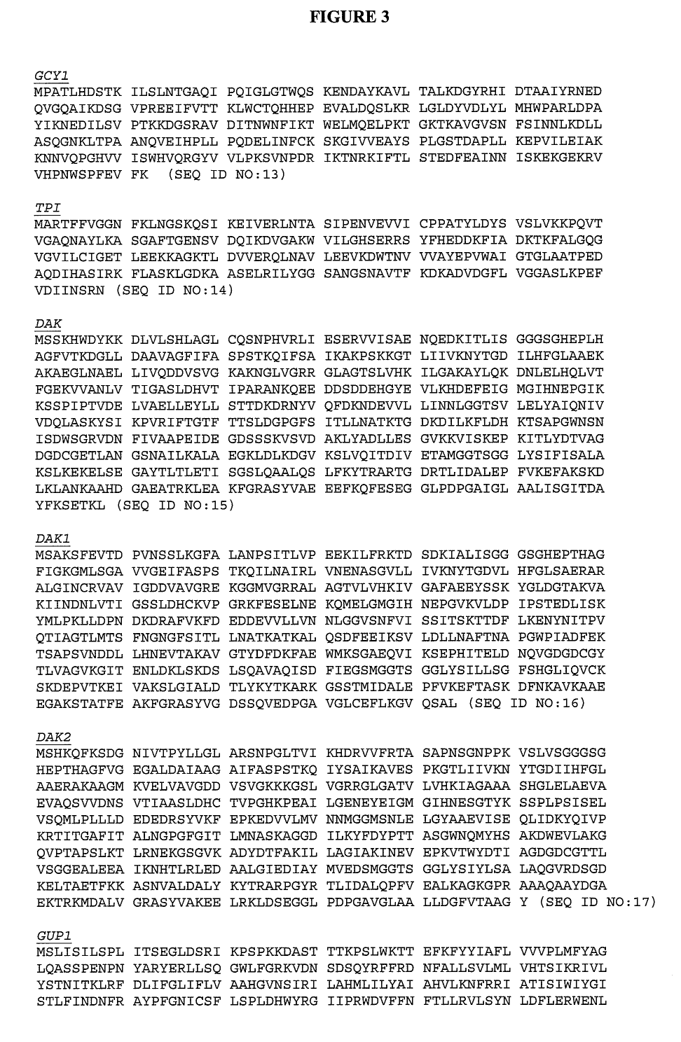

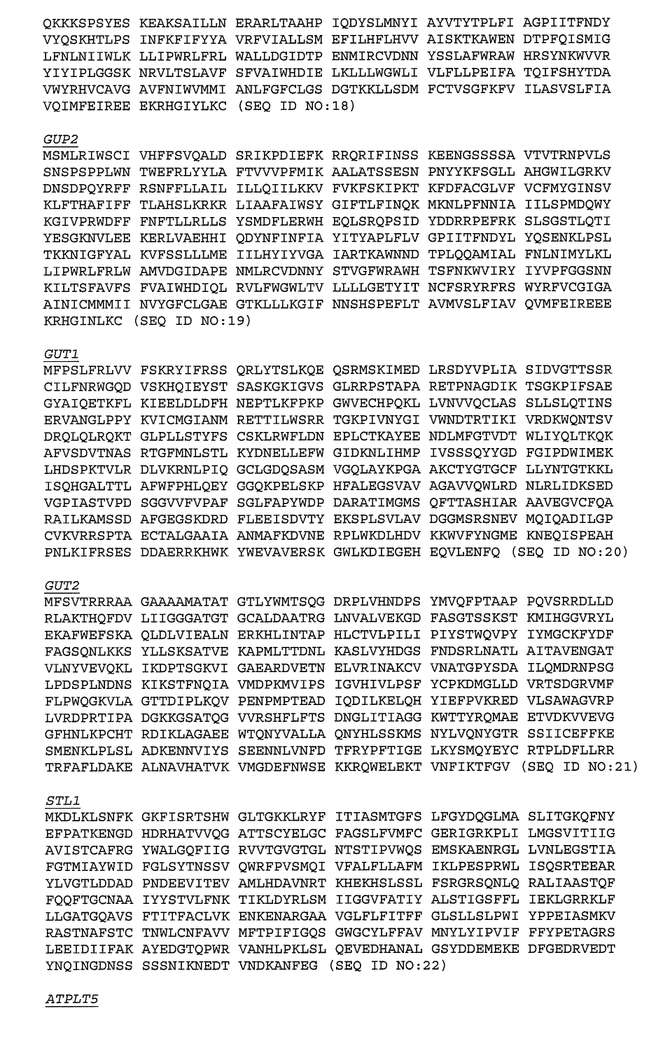

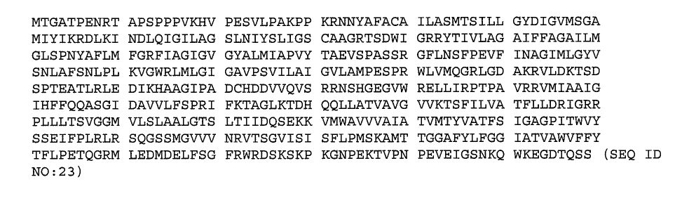

The yeast integrative (YI) pRS plasmid or other genetic methods used to integrate protein expression constructs into yeast chromosomes can be prepared using standard molecular biology methods. In some cases, a multiple restriction site region (MRSR) can be incorporated onto the ends of each insert to allow for further manipulation of the DNA. In some cases, the constructs contain a promoter operably linked to a coding sequence with at least 85%, 90%, or 95% identity to GCY1 (SEQ ID NO: 3), TPI (SEQ ID NO: 4), DAK (SEQ ID NO: 5), DAK1 (SEQ ID NO: 6), DAK2 (SEQ ID NO: 7), GUP1 (SEQ ID NO: 8), GUP2 (SEQ ID NO: 9), GUT1 (SEQ ID NO: 10), GUT2 (SEQ ID NO: 11), and/or STL1 (SEQ ID NO: 12) or wherein the coding sequence encodes a polypeptide at least 85%, 90%, or 95% identical to GCY1 (SEQ ID NO: 13), TPI (SEQ ID NO: 14), DAK (SEQ ID NO: 15), DAK1 (SEQ ID NO: 16), DAK2 (SEQ ID NO: 17), GUP1 (SEQ ID NO: 18), GUP2 (SEQ ID NO: 19), GUT1 (SEQ ID NO: 20), GUT2 (SEQ ID NO: 21), and/or STL1 (SEQ ID NO: 22). Each insert will contain a promoter (SEQ ID NO: 1) although the promoter can be any number of promoters that function in transcription and a yeast terminator such as the one presented with SEQ ID NO: 2. The nucleic acid sequence of each of the elements in the various inserts are shown in FIG. 2, and the amino acid sequence of the encoded polypeptides are shown in FIG. 3.

Once designed, constructs will be transformed into bacteria for propagation of the DNA material. Purified constructs (expression DNA inserts) can be transformed into target yeasts including, but not limited to, Fermax™ Gold Label and Fermax™ Green Label (Martrex, Inc.) and Ethanol Red (Fermentis). Construct can be sequentially inserted and this may be done without regard for order of insertion. After each successive transformation with a yeast insertion construct containing a gene related to glycerol conversion, individual colonies will be screened to ensure integration.

### Example 2

### Genetically-Engineered Yeast for Uptake and Conversion of Glycerol to Ethanol

A second yeast construct can be made that, in addition to the sequences described in Example 1 (+GCY1, +TPI, +DAK, +DAK1, +DAK2, +GUP1, +GUP2, +GUT1, +GUT2, and/or STL1), also contains the sequence with at least 85% identity for a polyol transporter ATPLT5 (SEQ ID NO: 23). The methods described in Example 1 can be used to make this second yeast construct.

It is to be understood that while the materials and methods have been described in conjunction with the detailed description thereof, the foregoing description is intended to illustrate and not limit the scope of the materials and methods, which is defined by the scope of the appended claims. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed. All publications, patents, and patent applications cited herein are hereby incorporated by reference in their entirety for all purposes.

1. A yeast comprising a first heterologous nucleic acid, a second heterologous nucleic acid, a third heterologous nucleic acid, and a fourth heterologous nucleic acid,
wherein expression of said first nucleic acid results in an increase in the amount or activity of a first polypeptide that is able to transport glycerol into the yeast cell;
wherein expression of said second nucleic acid results in an increase in the amount or activity of a second polypeptide that is able to convert glycerol into dihydroxyacetone (DHA);
wherein expression of said third nucleic acid results in an increase in the amount or activity of a third polypeptide that is able to phosphorylate DHA to produce dihydroxyacetone phosphate (DHAP); and
wherein expression of said fourth nucleic acid results in an increase in the amount or activity of a fourth polypeptide that is able to convert DHAP into glycereroldehyde-3-phosphase (GAP).

2. The yeast of claim 1, wherein said first polypeptide is a polyol transporter, a sugar transporter or a glycerol transporter, wherein said second polypeptide is a polypeptide from Enzyme Classification (EC) 1.1.99.22, wherein said third polypeptide is a polypeptide from EC 2.7.1.29, wherein said fourth polypeptide is a polypeptide from EC 5.3.1.1.

3. The yeast of claim 1,
wherein said first polypeptide is a polyol transporter, a sugar transporter or a glycerol transporter;
wherein said second polypeptide has glycerol dehydrogenase activity;
wherein said third polypeptide has dihydroxyacetone kinase activity; and
wherein said fourth polypeptide has triose-phosphate isomerase activity.

4. The yeast of claim 3, wherein said polypeptide having glycerol dehydrogenase activity has at least 85% sequence identity to SEQ ID NO:13.

5. The yeast of claim 3, wherein said polypeptide having a polyol transporter that has at least 85% sequence identity to SEQ ID NO:23.

6. The yeast of claim 3, wherein said polypeptide having dihydroxyacetone kinase activity has at least 85% sequence identity to SEQ ID NO: 16 or SEQ ID NO:17.

7. The yeast of claim 2, wherein said polypeptide having triosephosphate isomerase activity has at least 85% sequence identity to SEQ ID NO:14.

8. The yeast of claim 1, wherein said yeast is S. cerevisiae.

9. The yeast of claim 1, wherein, under fermentation conditions, said yeast produces reduced amounts of glycerol and increased amounts of ethanol compared to a yeast lacking a corresponding first, second, third, and/or fourth nucleic acid.

10. A method of producing ethanol, comprising: contacting the yeast of claim 1, with biomass, glycerol, carbohydrates and/or saccharides.

11.-12. (canceled)

13. A method of making the yeast of claim 1, the method comprising introducing the first nucleic acid, the second nucleic acid, the third nucleic acid, and the fourth nucleic acid into a yeast.

14. The method of claim 13, wherein said first nucleic acid encodes a polyol transporter, a sugar transporter or a glycerol transporter, wherein said second nucleic acid encodes a polypeptide having glycerol dehydrogenase activity, wherein said third nucleic acid encodes a polypeptide having dihydroxyacetone kinase activity, and wherein said fourth nucleic acid encodes a polypeptide having triose-phosphate isomerase activity.

15. The method of claim 13, wherein the yeast produces less glycerol and more ethanol than a corresponding yeast lacking the first, second, third and fourth nucleic acids.

16. The method of claim 13, wherein said first, second, third, or fourth nucleic acid sequences are integrated into the yeast chromosome.


SEQUENCE LISTING (24 sequences)

SEQ ID NO: 1 - DNA, 751 bp, Artificial Sequence
  synthetic promoter PGK
gctagccggccgcctaggaggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacagacgtcggtacc

SEQ ID NO: 2 - DNA, 180 bp, Artificial Sequence
  synthetic yeast terminator ADH1
gcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacc

SEQ ID NO: 3 - DNA, 939 bp, Saccharomyces cerevisiae
  glycerol dehydrogenase (GCY1)
atgcctgcgacactgcacgactccactaaaatactatccttgaataccggggcacaaatcccccagattggtttggggacttggcagtctaaagagaatgatgcttacaaggccgtgttaactgctttaaaagacggatacaggcacatagacactgccgcgatctatagaaacgaggatcaggtcggccaagcgatcaaggattcaggagtaccgagggaagaaatttttgtaaccacaaaattgtggtgtacacaacatcatgaaccagaagtagcccttgaccaatcactgaagagactaggtcttgactacgtagatttgtatcttatgcactggcctgcaaggctagatccagcttacataaagaatgaagatattctgtctgttccaactaaaaaggatggttccagagctgttgatattactaactggaacttcattaagacttgggaattgatgcaggagttgccaaagacaggcaaaaccaaggctgtaggtgtctctaatttcagtatcaacaatctgaaagaccttcttgcttcccaaggaaataagttgactcctgctgcaaaccaagttgagattcaccctttacttcctcaagatgaattgattaacttttgtaagagtaaaggtattgttgttgaggcctattctccattaggctctactgatgctccactactaaaagaacccgttatactagagatagcaaagaaaaacaatgtccaaccgggacatgttgtaatatcgtggcacgttcaacgtggatacgtagttcttcctaagtcggtcaatccagataggattaaaactaatagaaagatattcacattatctaccgaggactttgaagccatcaacaatattagcaaggaaaaaggtgaaaagagagtggtccatcctaattggtccccgtttgaagttttcaaatag

SEQ ID NO: 4 - DNA, 747 bp, Saccharomyces cerevisiae
  triosephosephate isomerase (TPI, TIM, TPIM)
atggctaggactttcttcgttggtggaaacttcaaattgaatggttctaaacaatcgattaaggaaattgtagaaagacttaatactgctagtattcctgaaaatgttgaagtggtaatttgtccaccagctacatatttggactacagtgtgtcacttgttaagaagcctcaggttaccgtaggtgcacaaaatgcttatttgaaggcatccggcgcctttacaggggagaattcggttgatcaaataaaagacgttggggcgaaatgggttattttgggtcatagcgaaaggagaagctatttccatgaggacgataagttcatagcagacaagacaaaattcgctttaggtcaaggtgtcggcgtgatattatgcataggggagacactagaagaaaagaaggctggtaagactcttgacgtggttgaacgtcagttgaatgctgtcttagaagaagttaaagattggactaatgtcgttgttgcttatgaaccggtctgggctataggtacgggcttggccgctacccctgaagatgcacaagatattcatgcttctatcagaaaattcctggcatctaagttgggtgataaggcagcgtcagaactgaggattttgtatggtggcagtgctaatggatcaaacgctgtcacattcaaggacaaggctgatgtggacggctttttagtcggtggagcctcattgaagcctgagtttgttgatatcataaactctcgtaattag

SEQ ID NO: 5 - DNA, 1827 bp, Saccharomyces cerevisiae
  dihydroxyacetone kinase (DAK), glycerone kinase
atgtcctcaaagcattgggattacaaaaaggatctagttctgtctcatttggcaggattatgtcaatcgaacccccacgttagactaattgagagcgaaagagttgttatttctgcagagaatcaggaggacaaaatcactttaattagtggaggcggttctggtcatgaaccactgcatgccggcttcgttacaaaagacggattactagacgcagctgtcgccggttttatcttcgcctcaccttccacaaagcaaattttctctgctatcaaagcaaagccatctaagaaaggtactttaatcattgttaagaattacacaggagatattttacacttcggattagctgcagaaaaggccaaggctgaaggactaaacgctgaattactaattgtccaggatgatgttagtgttggtaaagcgaaaaatggccttgttggtaggagaggcttagccggaacttcgttggttcataaaattctgggtgcgaaagcctatcttcaaaaggacaatcttgaactacatcaactagttacattcggggaaaaggtcgtcgccaatctggttacaattggtgcgtctttagatcacgtgacgattccagcaagggcaaataaacaagaggaggatgactccgatgatgaacatggttacgaagttttaaagcatgatgaatttgagataggaatgggtattcataatgaaccaggaatcaaaaagagctcgccaattcctaccgtcgatgaattggttgcagaattgctagaatatttgttgagcacgacagacaaagacagaaactatgttcaattcgataagaacgatgaggtagtcttactaataaacaatttaggtggaacatctgtccttgagctatatgccatccaaaacatcgttgtagatcaattggcgtctaagtatagcataaaaccagttagaattttcacgggcacctttactacgtcacttgacggtccgggtttttcaatcactctgttgaatgctaccaagacgggggataaggatatcttaaagtttctggatcataaaacttccgcacctggctggaactccaacatttctgattggtctggaagagttgacaactttattgtcgccgctcctgaaattgatgaaggcgattcctccagtaaagtgagcgtcgatgccaagttgtatgcagacttactagaatcaggtgtaaagaaggtaatttctaaggaaccaaaaattaccctttatgacactgtcgctggtgatggcgattgtggtgaaacattggctaatggtagtaacgctattctaaaggcattggctgaagggaaattggacttaaaggacggtgtgaagtccctagtgcaaattactgacatcgtagaaaccgctatgggtggtacatcaggcggcttgtattcgatttttatctcggctttagctaagagtttaaaagagaaggaactaagtgaaggggcatacacccttacattggaaaccatcagtggctcgcttcaagctgcgttacagtccttgttcaaatataccagagctagaactggtgacaggacattaatagatgctcttgagccattcgttaaagaattcgcaaagtctaaggacttaaaattggctaacaaggctgctcatgatggtgcagaagctactagaaaattggaagcaaaattcggtagggcatcatatgttgctgaagaggaattcaaacaatttgaatcggaaggagggcttccagatcccggtgccattggactagcggcattaatttctggcattactgatgcatacttcaaatcagaaaccaagttatag

SEQ ID NO: 6 - DNA, 1755 bp, Saccharomyces cerevisiae
  dihydroxyacetone kinase (DAK1), glycerone kinase
atgtcagccaagagctttgaagttaccgatcctgtaaattcctcactaaaaggtttcgcgctagctaatccctccattaccttagtccccgaggagaaaattttgtttcgtaaaacagattctgataagatcgcattaattagtggtggagggtccggtcacgagcccactcatgcaggtttcattggaaaaggaatgctgagcggtgctgtagttggagagatttttgcatcgccttccacaaaacaaatcttgaacgcgattcgtctggtaaacgaaaatgcgtcgggtgtattattgattgtcaagaactatactggtgacgttctgcattttggtttatccgccgaaagagcccgtgcacttggaatcaattgcagagtagctgttatcggcgatgacgtcgctgtcggtagggaaaaaggcggcatggtgggtagaagagcgttggcaggtacagttttggtgcataagatcgttggtgcattcgccgaggaatactcctctaagtacggtttagatggcaccgctaaagttgcaaagatcataaacgataacttggttacaatcggttcaagcttagatcattgtaaggtgccaggtagaaaatttgagtcagaactgaacgaaaaacaaatggaattgggtatgggtatccacaacgagccaggtgttaaagtactggacccaatccccagcactgaggacttaatttcaaaatacatgcttccgaagttgttagatcctaacgacaaggatagagcatttgtaaaatttgacgaagatgacgaagtagtgttattagtaaacaatttgggcggtgtatcaaattttgtgatttcaagtataaccagtaaaacaaccgatttcttgaaggaaaactacaatatcacaccagttcaaactatcgctggaactttaatgacctcctttaacggtaatggtttcagtattactcttcttaatgccaccaaagcgacaaaagccttgcaatcagatttcgaagaaataaagagtgtactggatcttttgaatgcgtttacgaatgctccaggctggcctattgccgattttgagaaaaccagtgcgcctagtgttaacgacgatctattacataacgaagttactgcaaaagcagtcggtacttatgattttgataagtttgcggagtggatgaaatccggcgctgaacaagtcattaaaagcgaaccccacataacagaattagacaaccaagttggcgatggtgattgcgggtatacactagtagccggtgtaaagggcatcacagaaaatctggacaaactatccaaggattcactttctcaagcagtcgcacaaatttctgatttcatcgaggggtccatgggtggaactagcggtggactatactcaatcttactgtctggtttctcccacggactgatccaggtttgcaaatccaaagacgaaccagtgacgaaggagatagtagccaaatccttgggtatcgctttggatactctgtacaagtatactaaggctagaaagggtagttcaacgatgatcgacgcacttgagccattcgttaaagagtttacagcttctaaagatttcaacaaagctgtcaaagctgctgaggaaggagcaaagagtacggcgactttcgaagctaagttcggaagagctagttatgttggtgacagctcacaagttgaagatcccggggctgtcggactttgtgaattcctgaaaggtgttcaatcagccttgtag

SEQ ID NO: 7 - DNA, 1776 bp, Saccharomyces cerevisiae
  dihydroxyacetone kinase (DAK2), glycerone kinase
atgtcccataagcaattcaaatcagacggtaatatagttacaccatacttattgggattggcaaggtcaaaccctggattgacggttataaagcatgacagagtagtgttccgtactgccagtgctcctaatagtggtaaccctcctaaagttagcttagtatctggcggtggcagcggtcatgaacctacacatgcaggctttgttggcgaaggtgcattggatgcgattgcagcaggcgctatcttcgcttctccttcaactaagcaaatatactcagcaattaaagcagttgaaagcccaaaaggaacactaataatcgtcaaaaactatacgggagacataatacatttcggtctagccgccgaacgtgctaaagcggctgggatgaaggttgagctagttgctgtgggtgacgatgtatctgttggcaaaaagaaaggttcccttgttggtaggagaggtttaggtgccactgtcttggtacataagattgccggggctgcagcttctcacggcttggaactggcggaagtcgccgaggttgctcagtcggtcgttgataattcggtcactattgctgctagtcttgatcattgtacggttccaggtcacaaaccagaagccatccttggagaaaatgagtatgagattggtatgggtatccacaacgaatctggtacatataagtctagccccttgccaagcatctcggagttagtctcccaaatgttaccactgcttttagatgaagatgaggaccgttcgtatgtcaaattcgaaccaaaagaagatgttgtattaatggtaaacaatatgggcggcatgagtaacttagagctgggttatgctgcagaggttatatccgaacaattaatcgataagtatcaaattgtaccaaagagaactattactggcgcgttcattacagctctaaacgggcctggttttggtataactttgatgaatgcatcaaaagcaggaggagatattctgaaatacttcgattacccaaccaccgcttctggatggaatcaaatgtatcattctgcaaaggattgggaggttttagccaagggtcaagttcctactgctccaagcctaaagactcttagaaacgaaaaaggttctggagttaaggctgattacgacacttttgctaaaatcttactggcaggaattgctaagattaatgaagtcgaacctaaagtcacctggtatgacacaattgccggtgatggggattgcggtactaccttggtttcaggtggagaggcattggaggaagctatcaagaatcatactttaagattagaagatgctgcactgggtattgaagatatcgcttacatggtagaagatagcatgggcggaaccagcggaggtttatattctatatacctgagcgccttggcgcaaggtgtcagggattctggtgataaagaattgacggcagaaacattcaaaaaggcaagcaacgtggctttagacgcattgtacaagtatactagggctagaccagggtacagaacgctaattgatgctttacaacctttcgttgaggctctgaaagctggtaagggtccaagggctgccgctcaagccgcttacgatggcgctgaaaagactagaaaaatggacgctctggttggaagagcaagttacgttgccaaagaagagttgagaaaattagacagtgaaggtgggttacccgatccaggggccgtgggcttggctgctttactagacggttttgttacagctgccggttattag

SEQ ID NO: 8 - DNA, 1683 bp, Saccharomyces cerevisiae
  glycerol uptake 1 (GUP1), glycerol transporter
atgagtttaataagcatactttcaccattgataaccagcgaaggtttggattccagaataaagccatcccccaagaaggatgcatctacgacaacgaagccttctctttggaaaacgactgagttcaagttctattacatagcttttctagtggttgttcctttaatgttttatgctggattacaggcatcaagtcctgaaaatcctaactacgcaaggtatgaaagactgttatctcaaggctggcttttcggacgtaaagtggataacagcgattcccaatataggtttttcagagataactttgctttgttaagtgttctaatgttggtccatacatctatcaaaagaattgtgttatactctactaacattacaaaacttaggttcgatttgatttttggcttaattttcttggtagctgcccatggtgtcaactcaatcagaatactggctcacatgctaatactgtatgctattgcacatgttttgaagaattttagacgtatagccactatttcaatctggatctatggtatctctacattgtttatcaatgataattttcgtgcatacccttttggtaatatctgtagctttttatccccattggatcattggtacagaggtattattcctagatgggacgtgtttttcaactttacattgttaagagttttaagctacaacttggatttcttggaaagatgggagaatctgcagaaaaagaaaagcccctcctacgagtctaaagaggctaagtcggctattttgttgaacgaaagggcaaggttaaccgctgcccatcctattcaagattattctttgatgaactatattgcttatgtgacttacacaccgctgtttattgctggcccgattatcacttttaacgactacgtatatcagtctaaacataccctgcccagtattaacttcaaattcattttctattacgcagttaggtttgtaatagctcttttgtccatggaattcatcttgcattttctacatgtggtagccattagtaaaacaaaggcatgggagaacgatacaccctttcaaatatccatgattggattgttcaatctaaacattatatggttgaaattactaattccctggagactattcagactttgggcattattggatgggatagatactccggaaaatatgatacgttgtgtcgataataactacagttcattggctttctggagagcatggcatagatcctataataagtgggtggtcaggtatatctacatcccactaggcggatctaaaaatagggttttaacttctttggctgtgttctctttcgtagctatttggcatgacatcgaattaaaacttttattgtggggctggcttattgtcttattccttcttccagaaatatttgctacgcaaattttctctcattacacagacgccgtttggtatcgtcatgtttgtgcagttggtgctgtgtttaacatttgggtgatgatgatagctaatctttttgggttttgcttaggtagtgacggtacaaagaagctattgtctgatatgttttgcactgtatcgggttttaagttcgtgatattggcctctgtaagcttgttcatagcagtccaaattatgtttgaaatcagagaggaggaaaagagacatggtatatacttaaaatgttag

SEQ ID NO: 9 - DNA, 1830 bp, Saccharomyces cerevisiae
  glycerol uptake 2 (GUP2), glycerol transporter
atgtcaatgttaagaatttggtcatgtattgttcatttctttagcgtacaagctttggatagtagaattaaaccagatatagaattcaagagaaggcagcgtatattcataaactcatccaaagaggagaatggctcatctagtagtgcggttacagtaactaggaacccggtgctgtcgagtaattcgccgagtccaccattgtggaacacctgggaattcaggctgtactacttagcattcacagttgtagtgccattcatgataaaagctgcactagcaacgtcgtccgaaagtaatccaaactattataagttctcaggccttcttgcccatggctggatattgggcagaaaggtagacaattctgacccacagtatagattctttcgttctaatttctttttgttagctatattgatccttttacaaattatcttaaagaaggttttcgtgaagttttctaaaattcccaagactaagtttgactttgcctgtggtctggttttcgtctgttttatgtatggtatcaatagcgtcaagctattcactcacgctttcatattctttaccctagctcactctcttaaaagaaagcgtctaattgctgcctttgcaatctggtcttatggaattttcactttgtttatcaatcaaaagatgaaaaatcttccctttaacaatatagccatcattttgagtccgatggaccaatggtacaagggcattgtacctagatgggacttctttttcaacttcactttgcttagattgttatcttattccatggatttcttggaacgttggcatgagcaattgtcccgtcagccttctatagattacgacgatcgtagacccgagtttagaaaatcactatcaggttcaacactacaaactatctatgagagtggtaagaatgtactagaagaaaaagagagattggttgctgaacatcatatccaagattacaacttcattaactttattgcatatatcacgtatgctcccctgtttttggttggaccaataataacattcaacgattacttatatcaaagcgaaaacaaactaccttccttgacgaaaaagaacattggcttttatgcgttaaaagtgttttcttcattacttcttatggagattatactacactacatatacgttggggcaatcgcaagaactaaagcctggaataatgatactccattacaacaagctatgattgcgttattcaatttgaatataatgtacttgaaactgcttataccctggagacttttcagattgtgggctatggttgacggaatcgatgctcctgaaaatatgttgagatgtgtggacaataactatagcacagtgggtttctggagagcttggcatacttcttttaacaagtgggtcataagatacatctacgtgccattcggtgggtccaacaacaaaatcttgacgtccttcgctgtattttcatttgttgccatttggcatgacattcagcttcgtgtcttgttttggggttggttaaccgtattgttattgttaggtgaaacttatattaccaattgcttttcgaggtatcgttttagatcatggtacagattcgtttgtgggattggtgcggcaattaacatttgcatgatgatgattattaacgtctatggcttttgtctgggtgctgaaggcacaaagctattactaaaaggtatattcaacaactcacactctccagagtttttaaccgcagttatggtcagtctattcatcgcggttcaagtcatgtttgaaataagagaagaagaaaagaggcatggtataaaccttaagtgctag

SEQ ID NO: 10 - DNA, 2127 bp, Artificial Sequence
  synthetic GUT1
atgtttccctctctcttccgacttgtagtattctccaaacgttacatattccgatcaagccagcgcctttacactagtttaaaacaagaacagagccgtatgtccaaaataatggaagatttacgaagtgactacgtcccgcttatcgccagtattgatgtaggaacgacctcatccagatgcattctgttcaacagatggggccaggacgtttcaaaacaccaaattgaatattcaacttcagcatcgaagggcaagattggggtgtctggcctaaggagaccctctacagccccagctcgtgaaacaccaaacgccggtgacatcaaaaccagcggaaagcccatcttttctgcagaaggctatgccattcaagaaaccaaattcctaaaaatcgaggaattggacttggacttccataacgaacccacgttgaagttccccaaaccgggttgggttgagtgccatccgcagaaattactggtgaacgtcgtccaatgccttgcctcaagtttgctctctctgcagactatcaacagcgaacgtgtagcaaacggtctcccaccttacaaggtaatatgcatgggtatagcaaacatgagagaaaccacaattctgtggtcccgccgcacaggaaaaccaattgttaactacggtattgtttggaacgacaccagaacgatcaaaatcgttagagacaaatggcaaaacactagcgtcgataggcaactgcagcttagacagaagactggattgccattgctctccacgtatttctcctgttccaagctgcgctggttcctcgacaatgagcctctgtgtaccaaggcgtatgaggagaacgacctgatgttcggcactgtggacacatggctgatttaccaattaactaaacaaaaggcgttcgtttctgacgtaaccaacgcttccagaactggatttatgaacctctccactttaaagtacgacaacgagttgctggaattttggggtattgacaagaacctgattcacatgcccgaaattgtgtcctcatctcaatactacggtgactttggcattcctgattggataatggaaaagctacacgattcgccaaaaacagtactgcgagatctagtcaagagaaacctgcccatacagggctgtctgggcgaccaaagcgcatccatggtggggcaactcgcttacaaacccggtgctgcaaaatgtacttatggtaccggttgctttttactgtacaatacggggaccaaaaaattgatctcccaacatggcgcactgacgactctagcattttggttcccacatttgcaagagtacggtggccaaaaaccagaattgagcaagccacattttgcattagagggttccgtcgctgtggctggtgctgtggtccaatggctacgtgataatttacgattgatcgataaatcagaggatgtcggaccgattgcatctacggttcctgattctggtggcgtagttttcgtccccgcatttagtggcctattcgctccctattgggacccagatgccagagccaccataatggggatgtctcaattcactactgcctcccacatcgccagagctgccgtggaaggtgtttgctttcaagccagggctatcttgaaggcaatgagttctgacgcgtttggtgaaggttccaaagacagggactttttagaggaaatttccgacgtcacatatgaaaagtcgcccctgtcggttctggcagtggatggcgggatgtcgaggtctaatgaagtcatgcaaattcaagccgatatcctaggtccctgtgtcaaagtcagaaggtctccgacagcggaatgtaccgcattgggggcagccattgcagccaatatggctttcaaggatgtgaacgagcgcccattatggaaggacctacacgatgttaagaaatgggtcttttacaatggaatggagaaaaacgaacaaatatcaccagaggctcatccaaaccttaagatattcagaagtgaatccgacgatgctgaaaggagaaagcattggaagtattgggaagttgccgtggaaagatccaaaggttggctgaaggacatagaaggtgaacacgaacaggttctagaaaacttccaa

SEQ ID NO: 11 - DNA, 1950 bp, Artificial Sequence
  synthetic GUT2
atgttttcagttactagaagaagagctgctggagccgctgctgctatggctaccgccactggtacactgtattggatgacctcccagggtgacagaccattggttcataatgacccctcctatatggttcaattccccacggctgcccctccacaggtaagcaggagggacttgttagatagactagctaaaacccaccaatttgatgtacttatcattggcggtggtgctacaggcactggttgcgcattggatgcagctacccgtggcttgaatgttgctttggttgagaaaggtgatttcgcatcaggtacaagcagtaagtctaccaaaatgatccatggtggagtccgttacttggaaaaagctttctgggaattcagcaaggcccaattagatttggtgatcgaagcattgaatgagcgtaaacacctaataaacactgctccacacctttgcactgtccttccgatcctgatcccgatatatagtacatggcaagttccatacatttacatgggctgcaaattctatgacttctttgctggtagtcaaaacttgaaaaagagttacttactgtcaaaatcggctactgtagaaaaggcccctatgttaacgacagataatcttaaggcctcgttagtttaccatgacggttcctttaatgactcgcgtcttaatgcgaccttggcaattacagcagtcgaaaacggtgccacggttctaaactatgtagaggttcagaagttgattaaggaccccacaagcggcaaggtaatcggtgctgaagccagggacgtagaaactaatgaattggttcgtattaatgctaagtgtgtagtcaacgcaactggtccttacagtgatgcaatattacaaatggataggaatcccagtggtttgcccgattccccattgaacgataattcgaaaattaagagtactttcaaccagatagcagttatggaccctaagatggttattccctctatcggtgtacatatcgttttacctagcttctactgtcctaaggacatgggattgctagacgtgagaaccagtgacggtagagtcatgtttttcctaccatggcagggtaaggtgttggctggtacaaccgatatccccctgaaacaagttccagaaaaccctatgcccactgaagccgatattcaagatattctgaaagaactacaacactacatcgaattcccggtcaagcgtgaagatgtcctgagcgcatgggcgggtgttcgtccattggtgcgtgatccaagaactattcctgctgacggtaaaaaggggagtgcaacgcaaggtgttgttagatcacattttctattcacaagtgacaatggtttgattacaatcgctggtggaaaatggacaacatatcgtcaaatggctgaagaaactgttgataaggttgtggaagtgggtgggtttcacaacttaaagccatgccatacaagagatataaagttggcaggtgctgaggaatggacacagaattacgtggccttgctagctcaaaattatcatttgtcatcaaagatgagcaattacttggtacaaaattatggtactaggagcagtataatttgtgaattctttaaggagtccatggaaaataagttgcccctatcgctggcagataaggaaaataatgtgatatattctagcgaagaaaacaacttagttaactttgatacctttaggtatccattcaccattggagaattaaagtactcaatgcaatatgaatattgtagaacgccgctagacttccttttaagaagaactagattcgcatttttagatgcaaaagaagccttgaatgctgtccatgcaactgtcaaagtcatgggtgatgagtttaattggagtgaaaagaaaagacaatgggaattggagaaaactgtcaacttcattaaaacttttggagtgtag

SEQ ID NO: 12 - DNA, 1710 bp, Saccharomyces cerevisiae
  sugar transporter (STL1)
atgaaagatttgaagttgtctaactttaaaggaaagtttatatcaaggacttcgcattggggtctaacgggtaagaagttaaggtactttatcactattgcttctatgacgggtttcagtctttttggttatgatcagggtttaatggcctcattaataaccggtaagcaattcaactatgaattcccggccacaaaagaaaatggggatcatgataggcatgcaactgttgtgcagggtgctactacatcctgttacgagctgggctgttttgccggttccctgttcgtgatgttctgcggtgaaaggatcggtagaaagccattgatacttatggggagcgtcataaccatcattggtgcagtaatttccacatgtgcctttagaggctactgggcacttggtcagttcataataggtagagttgtaaccggtgtgggtacgggactgaacacttctactatacctgtgtggcagtctgaaatgtcaaaagccgaaaacaggggtttattggtaaacctggaagggtcaacaatagcatttgggaccatgatagcatattggattgacttcggactttcgtacacaaatagctctgtacaatggagattcccagttagcatgcaaattgtctttgcgttattcctgttggccttcatgataaaattgccagagagtcctagatggcttatatcccagagcagaacggaagaggcaaggtatttggtcggcacattggacgacgccgatccaaacgatgaagaagtaattacggaggttgctatgttgcatgatgcagtaaacaggactaaacatgaaaagcactcactatctagtttgtttagcagaggcagatcccaaaacttacaaagggctttgatagctgcttcgacgcagttctttcaacaattcactggctgtaacgctgccatctactactcaacagttttgttcaacaaaactataaagttggattatagattgagcatgatcatcggcggtgtatttgcaactatctacgctttgtccactattggtagtttctttttgatcgaaaaattgggaaggagaaagctgttcctattgggtgctacaggacaagctgtgagtttcacaattaccttcgcctgtctagttaaagaaaacaaggagaacgcaagaggggccgcagtagggctttttctgtttataaccttctttggtttatccttgttatctttaccttggatctatcctccagaaatagcctcaatgaaggttagagcctccactaatgcattttcaacttgcaccaactggttatgcaattttgcagtcgtaatgttcacgccaattttcattggacagtctggttggggttgttatttgtttttcgctgtcatgaattatctttatatccccgtgatattcttcttttatccagagacagccggtagatcattagaagaaatagatattatatttgctaaggcatacgaggacggtactcaaccatggagagttgccaatcacttaccaaagctatccttacaagaagttgaagatcatgcaaatgcattggggagctatgacgatgaaatggaaaaagaagatttcggtgaggatagagtggaggatacgtacaatcaaattaatggtgacaatagtagctcatcgtctaatatcaagaacgaagatactgttaatgataaggctaatttcgaggggtag

SEQ ID NO: 13 - protein, 312 aa, Saccharomyces cerevisiae
  glycerol dehydrogenase (GCY1)
MPATLHDSTKILSLNTGAQIPQIGLGTWQSKENDAYKAVLTALKDGYRHIDTAAIYRNEDQVGQAIKDSGVPREEIFVTTKLWCTQHHEPEVALDQSLKRLGLDYVDLYLMHWPARLDPAYIKNEDILSVPTKKDGSRAVDITNWNFIKTWELMQELPKTGKTKAVGVSNFSINNLKDLLASQGNKLTPAANQVEIHPLLPQDELINFCKSKGIVVEAYSPLGSTDAPLLKEPVILEIAKKNNVQPGHVVISWHVQRGYVVLPKSVNPDRIKTNRKIFTLSTEDFEAINNISKEKGEKRVVHPNWSPFEVFK

SEQ ID NO: 14 - protein, 248 aa, Saccharomyces cerevisiae
  triosephosephate isomerase (TPI, TIM, TPIM)
MARTFFVGGNFKLNGSKQSIKEIVERLNTASIPENVEVVICPPATYLDYSVSLVKKPQVTVGAQNAYLKASGAFTGENSVDQIKDVGAKWVILGHSERRSYFHEDDKFIADKTKFALGQGVGVILCIGETLEEKKAGKTLDVVERQLNAVLEEVKDWTNVVVAYEPVWAIGTGLAATPEDAQDIHASIRKFLASKLGDKAASELRILYGGSANGSNAVTFKDKADVDGFLVGGASLKPEFVDIINSRN

SEQ ID NO: 15 - protein, 608 aa, Saccharomyces cerevisiae
  dihydroxyacetone kinase (DAK), glycerone kinase
MSSKHWDYKKDLVLSHLAGLCQSNPHVRLIESERVVISAENQEDKITLISGGGSGHEPLHAGFVTKDGLLDAAVAGFIFASPSTKQIFSAIKAKPSKKGTLIIVKNYTGDILHFGLAAEKAKAEGLNAELLIVQDDVSVGKAKNGLVGRRGLAGTSLVHKILGAKAYLQKDNLELHQLVTFGEKVVANLVTIGASLDHVTIPARANKQEEDDSDDEHGYEVLKHDEFEIGMGIHNEPGIKKSSPIPTVDELVAELLEYLLSTTDKDRNYVQFDKNDEVVLLINNLGGTSVLELYAIQNIVVDQLASKYSIKPVRIFTGTFTTSLDGPGFSITLLNATKTGDKDILKFLDHKTSAPGWNSNISDWSGRVDNFIVAAPEIDEGDSSSKVSVDAKLYADLLESGVKKVISKEPKITLYDTVAGDGDCGETLANGSNAILKALAEGKLDLKDGVKSLVQITDIVETAMGGTSGGLYSIFISALAKSLKEKELSEGAYTLTLETISGSLQAALQSLFKYTRARTGDRTLIDALEPFVKEFAKSKDLKLANKAAHDGAEATRKLEAKFGRASYVAEEEFKQFESEGGLPDPGAIGLAALISGITDAYFKSETKL

SEQ ID NO: 16 - protein, 584 aa, Saccharomyces cerevisiae
  dihydroxyacetone kinase (DAK1), glycerone kinase
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 17 - protein, 591 aa, Saccharomyces cerevisiae
  dihydroxyacetone kinase (DAK2), glycerone kinase
MSHKQFKSDGNIVTPYLLGLARSNPGLTVIKHDRVVFRTASAPNSGNPPKVSLVSGGGSGHEPTHAGFVGEGALDAIAAGAIFASPSTKQIYSAIKAVESPKGTLIIVKNYTGDIIHFGLAAERAKAAGMKVELVAVGDDVSVGKKKGSLVGRRGLGATVLVHKIAGAAASHGLELAEVAEVAQSVVDNSVTIAASLDHCTVPGHKPEAILGENEYEIGMGIHNESGTYKSSPLPSISELVSQMLPLLLDEDEDRSYVKFEPKEDVVLMVNNMGGMSNLELGYAAEVISEQLIDKYQIVPKRTITGAFITALNGPGFGITLMNASKAGGDILKYFDYPTTASGWNQMYHSAKDWEVLAKGQVPTAPSLKTLRNEKGSGVKADYDTFAKILLAGIAKINEVEPKVTWYDTIAGDGDCGTTLVSGGEALEEAIKNHTLRLEDAALGIEDIAYMVEDSMGGTSGGLYSIYLSALAQGVRDSGDKELTAETFKKASNVALDALYKYTRARPGYRTLIDALQPFVEALKAGKGPRAAAQAAYDGAEKTRKMDALVGRASYVAKEELRKLDSEGGLPDPGAVGLAALLDGFVTAAGY

SEQ ID NO: 18 - protein, 560 aa, Saccharomyces cerevisiae
  glycerol uptake 1 (GUP1), glycerol transporter
MSLISILSPLITSEGLDSRIKPSPKKDASTTTKPSLWKTTEFKFYYIAFLVVVPLMFYAGLQASSPENPNYARYERLLSQGWLFGRKVDNSDSQYRFFRDNFALLSVLMLVHTSIKRIVLYSTNITKLRFDLIFGLIFLVAAHGVNSIRILAHMLILYAIAHVLKNFRRIATISIWIYGISTLFINDNFRAYPFGNICSFLSPLDHWYRGIIPRWDVFFNFTLLRVLSYNLDFLERWENLQKKKSPSYESKEAKSAILLNERARLTAAHPIQDYSLMNYIAYVTYTPLFIAGPIITFNDYVYQSKHTLPSINFKFIFYYAVRFVIALLSMEFILHFLHVVAISKTKAWENDTPFQISMIGLFNLNIIWLKLLIPWRLFRLWALLDGIDTPENMIRCVDNNYSSLAFWRAWHRSYNKWVVRYIYIPLGGSKNRVLTSLAVFSFVAIWHDIELKLLLWGWLIVLFLLPEIFATQIFSHYTDAVWYRHVCAVGAVFNIWVMMIANLFGFCLGSDGTKKLLSDMFCTVSGFKFVILASVSLFIAVQIMFEIREEEKRHGIYLKC

SEQ ID NO: 19 - protein, 609 aa, Saccharomyces cerevisiae
  glycerol uptake 2 (GUP2), glycerol transporter
MSMLRIWSCIVHFFSVQALDSRIKPDIEFKRRQRIFINSSKEENGSSSSAVTVTRNPVLSSNSPSPPLWNTWEFRLYYLAFTVVVPFMIKAALATSSESNPNYYKFSGLLAHGWILGRKVDNSDPQYRFFRSNFFLLAILILLQIILKKVFVKFSKIPKTKFDFACGLVFVCFMYGINSVKLFTHAFIFFTLAHSLKRKRLIAAFAIWSYGIFTLFINQKMKNLPFNNIAIILSPMDQWYKGIVPRWDFFFNFTLLRLLSYSMDFLERWHEQLSRQPSIDYDDRRPEFRKSLSGSTLQTIYESGKNVLEEKERLVAEHHIQDYNFINFIAYITYAPLFLVGPIITFNDYLYQSENKLPSLTKKNIGFYALKVFSSLLLMEIILHYIYVGAIARTKAWNNDTPLQQAMIALFNLNIMYLKLLIPWRLFRLWAMVDGIDAPENMLRCVDNNYSTVGFWRAWHTSFNKWVIRYIYVPFGGSNNKILTSFAVFSFVAIWHDIQLRVLFWGWLTVLLLLGETYITNCFSRYRFRSWYRFVCGIGAAINICMMMIINVYGFCLGAEGTKLLLKGIFNNSHSPEFLTAVMVSLFIAVQVMFEIREEEKRHGINLKC

SEQ ID NO: 20 - protein, 708 aa, Artificial Sequence
  synthetic GUT1
MFPSLFRLVVFSKRYIFRSSQRLYTSLKQEQSRMSKIMEDLRSDYVPLIASIDVGTTSSRCILFNRWGQDVSKHQIEYSTSASKGKIGVSGLRRPSTAPARETPNAGDIKTSGKPIFSAEGYAIQETKFLKIEELDLDFHNEPTLKFPKPGWVECHPQKLLVNVVQCLASSLLSLQTINSERVANGLPPYKVICMGIANMRETTILWSRRTGKPIVNYGIVWNDTRTIKIVRDKWQNTSVDRQLQLRQKTGLPLLSTYFSCSKLRWFLDNEPLCTKAYEENDLMFGTVDTWLIYQLTKQKAFVSDVTNASRTGFMNLSTLKYDNELLEFWGIDKNLIHMPIVSSSQYYGDFGIPDWIMEKLHDSPKTVLRDLVKRNLPIQGCLGDQSASMVGQLAYKPGAAKCTYGTGCFLLYNTGTKKLISQHGALTTLAFWFPHLQEYGGQKPELSKPHFALEGSVAVAGAVVQWLRDNLRLIDKSEDVGPIASTVPDSGGVVFVPAFSGLFAPYWDPDARATIMGMSQFTTASHIARAAVEGVCFQARAILKAMSSDAFGEGSKDRDFLEEISDVTYEKSPLSVLAVDGGMSRSNEVMQIQADILGPCVKVRRSPTAECTALGAAIAANMAFKDVNERPLWKDLHDVKKWVFYNGMEKNEQISPEAHPNLKIFRSESDDAERRKHWKYWEVAVERSKGWLKDIEGEHEQVLENFQ

SEQ ID NO: 21 - protein, 649 aa, Artificial Sequence
  synthetic GUT2
MFSVTRRRAAGAAAAMATATGTLYWMTSQGDRPLVHNDPSYMVQFPTAAPPQVSRRDLLDRLAKTHQFDVLIIGGGATGTGCALDAATRGLNVALVEKGDFASGTSSKSTKMIHGGVRYLEKAFWEFSKAQLDLVIEALNERKHLINTAPHLCTVLPILIPIYSTWQVPYIYMGCKFYDFFAGSQNLKKSYLLSKSATVEKAPMLTTDNLKASLVYHDGSFNDSRLNATLAITAVENGATVLNYVEVQKLIKDPTSGKVIGAEARDVETNELVRINAKCVVNATGPYSDAILQMDRNPSGLPDSPLNDNSKIKSTFNQIAVMDPKMVIPSIGVHIVLPSFYCPKDMGLLDVRTSDGRVMFFLPWQGKVLAGTTDIPLKQVPENPMPTEADIQDILKELQHYIEFPVKREDVLSAWAGVRPLVRDPRTIPADGKKGSATQGVVRSHFLFTSDNGLITIAGGKWTTYRQMAEETVDKVVEVGGFHNLKPCHTRDIKLAGAEEWTQNYVALLAQNYHLSSKMSNYLVQNYGTRSSIICEFFKESMENKLPLSLADKENNVIYSSEENNLVNFDTFRYPFTIGELKYSMQYEYCRTPLDFLLRRTRFAFLDAKEALNAVHATVKVMGDEFNWSEKKRQWELEKTVNFIKTFGV

SEQ ID NO: 22 - protein, 569 aa, Saccharomyces cerevisiae
  sugar transporter (STL1)
MKDLKLSNFKGKFISRTSHWGLTGKKLRYFITIASMTGFSLFGYDQGLMASLITGKQFNYEFPATKENGDHDRHATVVQGATTSCYELGCFAGSLFVMFCGERIGRKPLILMGSVITIIGAVISTCAFRGYWALGQFIIGRVVTGVGTGLNTSTIPVWQSEMSKAENRGLLVNLEGSTIAFGTMIAYWIDFGLSYTNSSVQWRFPVSMQIVFALFLLAFMIKLPESPRWLISQSRTEEARYLVGTLDDADPNDEEVITEVAMLHDAVNRTKHEKHSLSSLFSRGRSQNLQRALIAASTQFFQQFTGCNAAIYYSTVLFNKTIKLDYRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKEDFGEDRVEDTYNQINGDNSSSSSNIKNEDTVNDKANFEG

SEQ ID NO: 23 - protein, 539 aa, Artificial Sequence
  synthetic polyol transporter ATPLT5
MTGATPENRTAPSPPPVKHVPESVLPAKPPKRNNYAFACAILASMTSILLGYDIGVMSGAMIYIKRDLKINDLQIGILAGSLNIYSLIGSCAAGRTSDWIGRRYTIVLAGAIFFAGAILMGLSPNYAFLMFGRFIAGIGVGYALMIAPVYTAEVSPASSRGFLNSFPEVFINAGIMLGYVSNLAFSNLPLKVGWRLMLGIGAVPSVILAIGVLAMPESPRWLVMQGRLGDAKRVLDKTSDSPTEATLRLEDIKHAAGIPADCHDDVVQVSRRNSHGEGVWRELLIRPTPAVRRVMIAAIGIHFFQQASGIDAVVLFSPRIFKTAGLKTDHQQLLATVAVGVVKTSFILVATFLLDRIGRRPLLLTSVGGMVLSLAALGTSLTIIDQSEKKVMWAVVVAIATVMTYVATFSIGAGPITWVYSSEIFPLRLRSQGSSMGVVVNRVTSGVISISFLPMSKAMTTGGAFYLFGGIATVAWVFFYTFLPETQGRMLEDMDELFSGFRWRDSKSKPKGNPEKTVPNPEVEIGSNKQWKEGDTQSS

SEQ ID NO: 24 - protein, 6 aa, Artificial Sequence
  synthetic 6xHis purification tag
HHHHHH